(19) United States
(12) Patent Application Publication
(10) Pub. No.: US 2023/0241029 A1
(43) Pub. Date: Aug. 3, 2023

(54) METHODS OF REDUCING POST-SURGICAL METASTASIS

(71) Applicants: Kristoffer Hellstrand, Gothenburg (SE); Anna Martner, Gothenburg (SE)

(72) Inventors: Kristoffer Hellstrand, Gothenburg (SE); Anna Martner, Gothenburg (SE)

(21) Appl. No.: 18/002,963

(22) PCT Filed: Dec. 3, 2019
(86) PCT No.: PCT/US19/64151

§ 371 Date: Dec. 22, 2022

Related U.S. Application Data
(60) Provisional application No. 62/778,163, filed on Dec. 11, 2018.

(51) Int. Cl. A61K 31/417; A61P 35/04; A61P 41/00; A61P 29/00

(52) U.S. Cl. CPC A61K 31/417; A61P 35/04; A61P 41/00; A61P 29/00

(57) ABSTRACT

Some embodiments of the methods and compositions provided herein include methods of reducing surgery-induced metastases in a subject with cancer following surgery to remove at least one solid tumor, comprising an NADPH oxidase inhibitor to said subject.

### CROSS-REFERENCE TO RELATED APPLICATIONS

This application claims priority to U.S. Prov. App. No. 62/778,163 filed Dec. 11, 2018 entitled “METHODS OF REDUCING POST-SURGICAL METASTASIS” the entire contents of which is incorporated by reference in its entirety.

### FIELD OF THE INVENTION

Some embodiments provided herein relate to methods and compositions of using a NADPH oxidase inhibitor to reduce surgery- or wounding-induced inflammation, reduce reactive oxygen species, reduce the risk of metastases and reduce surgery-induced metastases in a subject.

### BACKGROUND OF THE INVENTION

The trauma associated with cancer surgery may result in a release of tumor cells into the blood stream along with a systemic inflammatory reaction that hampers cellular immunity. Both of these factors may enhance the risk of establishment of distant metastases. During infection, inflammation and cancer, myeloid cells, including granulocytes, monocytes and tissue-resident macrophages produce enhanced levels of a NADPH oxidase-derived (such as NOX2) reactive oxygen species (ROS). In addition to contributing to pathogen clearance, NADPH oxidase-derived ROS released from myeloid cells may trigger dysfunction and apoptosis in adjacent anti-neoplastic lymphocytes. Thus, there has been an unmet need for efficient methods and compositions of reducing the risk of metastatic disease associated with the surgical removal of solid tumors.

### SUMMARY OF THE INVENTION

Some embodiments of the methods and compositions provided herein include methods of reducing surgery-induced inflammation in a subject with cancer following surgery to remove at least one solid tumor, comprising administering an effective amount of an NADPH oxidase inhibitor to the subject.

In some embodiments, the inflammation the subject is reduced.

In some embodiments, the level of reactive oxygen species in the subject is reduced.

In some embodiments, the subject is human.

In some embodiments, the NADPH oxidase inhibitor is selected from the group consisting of histamine dihydrochloride, GSK2795039, apocynin, GKT136901, GKT137831, ML171, VAS2870, VAS3947, celastrol, ebselen, perhexiline, grindelic acid, NOX2ds-tat, NOXA1ds, fulvene-5, ACD 084, shionogi, histamine, N-methyl-histamine, 4-methyl-histamine, histamine phosphate, histamine diphosphate, a RAC1 inhibitor, a RAC2 inhibitor, NSC23766, CAS 1177865-17-6, CAS 1090893-12-1, idelalisib, and a class I phosphatidylinositol 3 kinase delta (PI3K delta) inhibitor.

In some embodiments, the NADPH oxidase inhibitor is histamine dihydrochloride or N-alpha-methyl-histamine dihydrochloride.

In some embodiments, the NADPH oxidase inhibitor is histamine dihydrochloride.

In some embodiments, the NADPH oxidase inhibitor is administered prior to, during or after surgery or a combination thereof.

In some embodiments, the cancer comprises a melanoma.

Some embodiments of the methods and compositions provided herein include methods of reducing reactive oxygen species in a subject with cancer following surgery to remove at least one solid tumor, comprising administering an effective amount of an NADPH oxidase inhibitor to the subject.

Some embodiments of the methods and compositions provided herein include methods of reducing the risk of metastases in a subject with cancer following surgery to remove at least one solid tumor, comprising administering an effective amount of an NADPH oxidase inhibitor to the subject.

Some embodiments of the methods and compositions provided herein include methods of reducing surgery-induced metastases in a subject with cancer following surgery to remove at least one solid tumor, comprising administering an NADPH oxidase inhibitor to the subject.

Some embodiments of the methods and compositions provided herein include methods of reducing wounding-induced inflammation in a subject following wounding, comprising administering an effective amount of an NADPH oxidase inhibitor to the subject. In some embodiments, the wounding is associated with surgery.

In some embodiments, the cancer comprises a melanoma.

Some embodiments of the methods and compositions provided herein include methods of reducing surgery-induced inflammation in a subject, comprising: administering an effective amount of an NADPH oxidase inhibitor to the subject.

In some embodiments, the inflammation is reduced compared to a subject having surgery and not administered the NADPH oxidase inhibitor.

In some embodiments, the subject has a metastatic cancer.

In some embodiments, the administering an effective amount of an NADPH oxidase inhibitor to the subject is sufficient to reduce metastasis of the metastatic cancer compared to a subject having a metastatic cancer and not administered the NADPH oxidase inhibitor.

In some embodiments, the metastatic cancer comprises a melanoma.

In some embodiments, the NADPH oxidase inhibitor is administered prior to the surgery.

In some embodiments, the NADPH oxidase inhibitor is administered after the surgery.

In some embodiments, the NADPH oxidase inhibitor is administered at least weekly.

In some embodiments, the NADPH oxidase inhibitor is administered at least three times weekly.

In some embodiments, the NADPH oxidase inhibitor is administered for at least 2 weeks.

In some embodiments, the subject is human.

In some embodiments, the NADPH oxidase inhibitor is selected from the group consisting of histamine dihydrochloride, GSK2795039, apocynin, GKT136901, GKT137831, ML171, VAS2870, VAS3947, celastrol, ebselen, perhexiline, grindelic acid, NOX2ds-tat, NOXA1ds, fulvene-5, ACD 084, shionogi, histamine, N-methyl-histamine, 4-methyl-histamine, histamine phosphate, histamine diphosphate, a RAC1 inhibitor, a RAC2 inhibitor, NSC23766, CAS 1177865-17-6, CAS 1090893-12-1, idelalisib, and a class I phosphatidylinositol 3 kinase delta (PI3K delta) inhibitor. In some embodiments, the NADPH oxidase inhibitor is histamine dihydrochloride or N-alpha-methyl-histamine dihydrochloride. In some embodiments, the NADPH oxidase inhibitor is histamine dihydrochloride. In some embodiments, the NADPH oxide inhibitor is a NOX2 inhibitor.

### BRIEF DESCRIPTION OF THE DRAWINGS

FIG. 1A depicts the formation of metastatic foci in the lungs. Surgical wounding increases the formation of metastases in a NOX2-dependent manner. Wild type or Nox2−/− mice were subjected to implantation of subcutaneous PVA sponges to induce tissue inflammation (INFL) (A) One week after implantation of sponges B16F10 melanoma cells were injected intravenously (100,000 to wild type mice and 150,000 to NOX2−/− mice). Metastatic foci formed in lungs were enumerated macroscopically after 3 weeks.

FIG. 1B depicts the presence of inflammatory monocytes in the blood. One week after implantation of sponges, blood was analyzed for content of CD11b+LyC6+inflammatory monocytes (infl. mono.). The values were normalized against those of unwounded control mice on each collection day.

FIG. 1C depicts ROS levels in inflammatory monocytes as determined by DCFDA staining one week after sponge implantation. The values were normalized against those of unwounded control mice on each collection day.

### DETAILED DESCRIPTION

Some embodiments provided herein relate to therapies for treating/ameliorating a cancer. Some therapies for treating a cancer can include the surgical removal of a tumor. During surgery, malignant cells can be unavoidably released into the circulation of a subject, meaning that subjects undergoing surgery for solid tumors are concomitantly potentially cured, but also exposed to a health threat from the released malignant cells. Surgery also entails inflammation, resulting from the surgical procedure itself. Such inflammation and/or surgery can induce a surgical stress which can create an increased risk factor for metastasis. See e.g., Krall J. A. et al. (2018) Sci Transl Med. 10(436). pii: eaan3464. doi: 10.1126/scitranslmed.aan3464 which is incorporated by reference in its entirety. As provided herein, a surgical procedure markedly elevated the risk of distant metastasis. Also demonstrated herein is the finding that triggering of NOX2 during surgical stress, as judged from results obtained in animals that genetically lack NOX2, was likely a factor that contributed to the increased risk of metastasis. It was also shown that the pharmacological inhibition of NOX2 (using HDC) reduced the surgery-induced inflammation and reduced the surgery-induced metastasis.

Some embodiments of the methods and compositions provided herein include methods of reducing surgery-induced inflammation in a subject with cancer following surgery to remove at least one solid tumor, comprising administering an effective amount of an NADPH oxidase inhibitor to the subject.

Some embodiments of the methods and compositions provided herein include methods of reducing reactive oxygen species in a subject with cancer following surgery to remove at least one solid tumor, comprising administering an effective amount of an NADPH oxidase inhibitor to the subject.

Some embodiments of the methods and compositions provided herein include methods of reducing the risk of metastases in a subject with cancer following surgery to remove at least one solid tumor, comprising administering an effective amount of an NADPH oxidase inhibitor to the subject.

Some embodiments of the methods and compositions provided herein include methods of reducing surgery-induced metastases in a subject with cancer following surgery to remove at least one solid tumor, comprising administering an effective amount of an NADPH oxidase inhibitor to the subject.

Some embodiments of the methods and compositions provided herein include methods of reducing wounding-induced inflammation in a subject following wounding, comprising administering an effective amount of an NADPH oxidase inhibitor to the subject.

NADPH oxidase (NOX) isoforms have multiple functions important for normal physiology and have been implicated in the pathogenesis of a broad range of diseases associated with inflammation, including atherosclerosis, cancer and neurodegenerative diseases.

NADPH oxides include NADPH oxidase 2 (NOX2). NOX2 is also known as cytochrome b(558) subunit beta or cytochrome b-245 heavy chain and is a protein that in humans is encoded by the NOX2 gene (also called CYBB gene). The protein is a superoxide generating enzyme which forms reactive oxygen species (ROS).

Reactive oxygen species are often produced by the incomplete reduction of oxygen. The complete reduction of one molecule of oxygen to water is a four-electron process. Oxidative metabolism continually generates partially reduced species of oxygen, which are far more reactive, and hence more toxic than oxygen itself. A one-electron reduction of oxygen yields superoxide ion (O²⁻); reduction by an additional electron yields hydrogen peroxide (H2O2), and reduction by a third electron yields a hydroxyl radical, and a hydroxide ion. Hydroxyl radicals in particular are extremely reactive and represent the most active mutagen derived from ionizing radiation. Each of these species are generated and must be converted to less reactive compounds to avoid tissue damage.

As discussed herein, particular cells of the immune system have harnessed the toxic effects of ROS as an effector mechanism. Phagocytic cells of the myeloid lineage such as polymorphonuclear leukocytes (neutrophils, PMN), monocytes, macrophages, and eosinophils function to protect the host in which they reside from infection by seeking out and destroying invading microbes. These phagocytic cells possess a membrane-bound enzyme system, the NADPH oxidase, which can be activated to produce toxic oxygen radicals in response to a wide variety of stimuli.

As used herein, “cancer” means primary and metastatic malignant solid tumor disease. Example cancers include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas cystadenocarcinoma, medullary carcinoma, bronchogenic carcinoma, renal cell carcinoma, hepatoma bile duct carcinoma choriocarcinoma seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme astrocytoma medulloblastoma, craniopharyngioma, ependymoma, pinealoma, hemangioblastoma acoustic neuroma, oligodendroglioma, meningioma, skin cancer, melanoma, neuroblastoma, and retinoblastoma.

In some embodiments, an effective amount of a NOX2 inhibitor can be administered to a subject in need thereof. Examples of NOX2 inhibitors include histamine dihydrochloride (HDC) (CEPLENE), GSK2795039, apocynin, GKT136901, GKT137831, ML171, VAS2870, VAS3947, celastrol, ebselen, perhexiline, grindelic acid, NOX2ds-tat, NOXAlds, fulvene-5, ACD 084, and shionogi. Additional examples of NOX2 inhibitors include histamine, N-methyl-histamine, 4-methyl-histamine, histamine phosphate, and histamine diphosphate. In some embodiments, the NOX2 inhibitor is HDC.

In some embodiments, a NOX2 inhibitor can include RAC1 inhibitors and RAC2 inhibitor, such as NSC23766, CAS 1177865-17-6, and CAS 1090893-12-1. RAC1 and RAC2 can each be associated with NOX2 holoenzyme, and inhibition of RAC1 or RAC 2 can inhibit NOX2. More examples of NOX2 inhibitors include idelaisib and other inhibitors of class I phosphatidylinositol 3 kinase delta (PI3K delta). Condliffe, A. M., K. et al., (2005) “Sequential activation of class IB and class IA PI3K is important for the primed respiratory burst of human but not murine neutrophils” Blood 106: 1432-1440 which is incorporated by reference in its entirety.

### Certain Methods

Some embodiments of the methods and compositions provided herein include methods of reducing surgery-induced inflammation in a subject with a cancer following surgery to remove at least one solid tumor, comprising administering an effective amount of an NADPH oxidase inhibitor to the subject. In some embodiments, the inflammation is reduced compared to a subject with a cancer having surgery and not administered the NADPH oxidase inhibitor. Some embodiments also include reducing reactive oxygen species in the subject, in which the effective amount of an NADPH oxidase inhibitor is sufficient to reduce reactive oxygen species in the subject compared to a subject not administered the NADPH oxidase inhibitor. In some embodiments, the subject is mammalian. In some embodiments, the subject is human. In some embodiments, the NADPH oxidase inhibitor is selected from the group consisting of histamine dihydrochloride, GSK2795039, apocynin, GKT136901, GKT137831, ML171, VAS2870, VAS3947, celastrol, ebselen, perhexiline, grindelic acid, NOX2ds-tat, NOXA1ds, fulvene-5, ACD 084, shionogi, histamine, N-methyl-histamine, 4-methyl-histamine, histamine phosphate, histamine diphosphate, a RAC1 inhibitor, a RAC2 inhibitor, NSC23766, CAS 1177865-17-6, CAS 1090893-12-1, idelalisib, and a class I phosphatidylinositol 3 kinase delta (PI3K delta) inhibitor. In some embodiments, the NADPH oxidase inhibitor is histamine dihydrochloride or N-alpha-methyl-histamine dihydrochloride. In some embodiments, the NADPH oxidase inhibitor is histamine dihydrochloride. In some embodiments, the NADPH oxide inhibitor is a NOX2 inhibitor. In some embodiments, the NADPH oxidase inhibitor is administered prior to the surgery. In some embodiments, the NADPH oxidase inhibitor is administered during the surgery. In some embodiments, the NADPH oxidase inhibitor is administered after the surgery. In some embodiments, the NADPH oxidase inhibitor is administered daily, weekly, or monthly. In some embodiments, the NADPH oxidase inhibitor is administered at least daily, at least weekly, or at least monthly. In some embodiments, the NADPH oxidase inhibitor is administered at once, twice, or three times weekly. In some embodiments, the NADPH oxidase inhibitor is administered at least once, twice, or three times weekly. In some embodiments, the NADPH oxidase inhibitor is administered for at least 1, 2, 3, 4, 5, 6 weeks, or for a period between any two of the foregoing numbers.

Some embodiments of the methods and compositions provided herein include methods of reducing reactive oxygen species in a subject with cancer following surgery to remove at least one solid tumor, comprising administering an effective amount of an NADPH oxidase inhibitor to the subject. Some embodiments of the methods and compositions provided herein include methods of reducing the risk of metastases in a subject with cancer following surgery to remove at least one solid tumor, comprising administering an effective amount of an NADPH oxidase inhibitor to the subject. Some embodiments of the methods and compositions provided herein include methods of reducing surgery-induced metastases in a subject with cancer following surgery to remove at least one solid tumor, comprising administering an NADPH oxidase inhibitor to the subject. Some embodiments of the methods and compositions provided herein include methods of reducing wounding-induced inflammation in a subject following wounding, comprising administering an effective amount of an NADPH oxidase inhibitor to the subject. In some embodiments, the wounding is associated with surgery. In some embodiments, the NADPH oxidase inhibitor is administered prior to the surgery. In some embodiments, the NADPH oxidase inhibitor is administered during the surgery. In some embodiments, the NADPH oxidase inhibitor is administered after the surgery. In some embodiments, the NADPH oxidase inhibitor is administered daily, weekly, or monthly. In some embodiments, the NADPH oxidase inhibitor is administered at least daily, at least weekly, or at least monthly. In some embodiments, the NADPH oxidase inhibitor is administered at once, twice, or three times weekly. In some embodiments, the NADPH oxidase inhibitor is administered at least once, twice, or three times weekly. In some embodiments, the NADPH oxidase inhibitor is administered for at least 1, 2, 3, 4, 5, 6 weeks, or for a period between any two of the foregoing numbers. In some embodiments, the cancer comprises a melanoma. In some embodiments, the subject is mammalian. In some embodiments, the subject is human. In some embodiments, the NADPH oxidase inhibitor is selected from the group consisting of histamine dihydrochloride, GSK2795039, apocynin, GKT136901, GKT137831, ML171, VAS2870, VAS3947, celastrol, ebselen, perhexiline, grindelic acid, NOX2ds-tat, NOXA1ds, fulvene-5, ACD 084, shionogi, histamine, N-methyl-histamine, 4-methyl-histamine, histamine phosphate, histamine diphosphate, a RAC1 inhibitor, a RAC2 inhibitor, NSC23766, CAS 1177865-17-6, CAS 1090893-12-1, idelalisib, and a class I phosphatidylinositol 3 kinase delta (PI3K delta) inhibitor. In some embodiments, the NADPH oxidase inhibitor is histamine dihydrochloride or N-alpha-methyl-histamine dihydrochloride. In some embodiments, the NADPH oxidase inhibitor is histamine dihydrochloride. In some embodiments, the NADPH oxide inhibitor is a NOX2 inhibitor.

Some embodiments of the methods and compositions provided herein include methods of reducing surgery-induced inflammation in a subject, comprising: administering an effective amount of an NADPH oxidase inhibitor to the subject. In some embodiments, the inflammation is reduced compared to a subject having surgery and not administered the NADPH oxidase inhibitor. In some embodiments, the subject has a metastatic cancer. In some embodiments, the administering an effective amount of an NADPH oxidase inhibitor to the subject is sufficient to reduce metastasis of the metastatic cancer compared to a subject having a metastatic cancer and not administered the NADPH oxidase inhibitor. In some embodiments, the metastatic cancer comprises a melanoma. In some embodiments, the NADPH oxidase inhibitor is administered prior to the surgery. In some embodiments, the NADPH oxidase inhibitor is administered during the surgery. In some embodiments, the NADPH oxidase inhibitor is administered after the surgery. In some embodiments, the NADPH oxidase inhibitor is administered daily, weekly, or monthly. In some embodiments, the NADPH oxidase inhibitor is administered at least daily, at least weekly, or at least monthly. In some embodiments, the NADPH oxidase inhibitor is administered at once, twice, or three times weekly. In some embodiments, the NADPH oxidase inhibitor is administered at least once, twice, or three times weekly. In some embodiments, the NADPH oxidase inhibitor is administered for at least 1, 2, 3, 4, 5, 6 weeks, or for a period between any two of the foregoing numbers. In some embodiments, the cancer comprises a melanoma. In some embodiments, the subject is mammalian. In some embodiments, the subject is human. In some embodiments, the NADPH oxidase inhibitor is selected from the group consisting of histamine dihydrochloride, GSK2795039, apocynin, GKT136901, GKT137831, ML171, VAS2870, VAS3947, celastrol, ebselen, perhexiline, grindelic acid, NOX2ds-tat, NOXA1ds, fulvene-5, ACD 084, shionogi, histamine, N-methyl-histamine, 4-methyl-histamine, histamine phosphate, histamine diphosphate, a RAC1 inhibitor, a RAC2 inhibitor, NSC23766, CAS 1177865-17-6, CAS 1090893-12-1, idelalisib, and a class I phosphatidylinositol 3 kinase delta (PI3K delta) inhibitor. In some embodiments, the NADPH oxidase inhibitor is histamine dihydrochloride or N-alpha-methyl-histamine dihydrochloride. In some embodiments, the NADPH oxidase inhibitor is histamine dihydrochloride. In some embodiments, the NADPH oxide inhibitor is a NOX2 inhibitor.

The following Examples further illustrate certain embodiments and are not to be construed as limiting its scope in any way.

### Methods of Treatment

Some embodiments of the methods and compositions provided herein include preventing, treating or ameliorating a subject having a cancer, such as a colon cancer, or a breast cancer. As used herein, “subject” can include a human or a non-human mammal, e.g., a dog, a cat, a mouse, a rat, a cow, a sheep, a pig, a goat, a non-human primate or a bird, e.g., a chicken, as well as any other vertebrate or invertebrate. As used herein, “treat,” “treatment,” or “treating,” can include administering a pharmaceutical composition to a subject for therapeutic purposes, and can include reducing the symptoms or consequences of a disorder, such as preventing the occurrence of a colon or breast tumor, reducing the number of tumor cells of a colon or breast tumor or inhibiting the growth of tumor cells of a colon or breast tumor; and can include curing a disorder, such as eliminating the symptoms of a disorder, such as the elimination of colon or breast tumor cells in a subject. As used herein, “ameliorate”, or “ameliorating” can include a therapeutic effect which relieves, to some extent, one or more of the symptoms of a disorder. As used herein, “prevent,” “preventing” and “prevention” can include inhibiting the occurrence of a disorder, and can include preventing a an action that occurs before a subject begins to suffer from the regrowth of the cancer and/or which inhibits or reduces the severity of the cancer. As used herein, an “effective amount” can include an amount, such as a dose, of a therapeutic compound sufficient to treat a disorder. As used herein, reducing the activity of NOX2 can include reducing the activity of NADPH oxidase 2, and/or reducing the activity of a NADPH oxidase holoenzyme which includes the NOX2 protein.

Some embodiments include reducing the activity of NOX2 by administering to a subject an agent that reduces the activity of NOX2 in the cell of the subject, such as a NOX2 inhibitor. In some embodiments, an effective amount of a NOX2 inhibitor can be administered to a subject in need thereof. Examples of NOX2 inhibitors include histamine dihydrochloride (HDC) (CEPLENE), GSK2795039, apocynin, GKT136901, GKT137831, ML171, VAS2870, VAS3947, celastrol, ebselen, perhexiline, grindelic acid, NOX2ds-tat, NOXAlds, fulvene-5, ACD 084, and shionogi. Altenhofer, S. et al., “Evolution of NADPH Oxidase Inhibitors: Selectivity and Mechanisms for Target Engagement”, Antioxid Redox Signal. 2015 23: 406-427; Hirano, K. et al., “Discovery of GSK2795039, a Novel Small Molecule NADPH Oxidase 2 Inhibitor”, Antioxid Redox Signal. 2015 23: 358-374, which are each incorporated by reference in its entirety. More examples of NOX2 inhibitors include histamine, N-methyl-histamine, 4-methyl-histamine, histamine phosphate, histamine diphosphate, idelalisib, and a class I phosphatidylinositol 3 kinase delta (PI3K delta) inhibitor. In some embodiments, the NOX2 inhibitor is HDC.

In some embodiments, a NOX2 inhibitor can include RAC1 inhibitors and RAC2 inhibitor, such as NSC23766, CAS 1177865-17-6, and CAS 1090893-12-1. RAC1 and RAC2 can each be associated with NOX2 holoenzyme, and inhibition of RAC1 or RAC 2 can inhibit NOX2. See e.g., Veluthakal R., et al., (2016) “NSC23766, a Known Inhibitor of Tiam1-Rac1 Signaling Module, Prevents the Onset of Type 1 Diabetes in the NOD Mouse Model” Cell Physiol Biochem 39:760-767; and Cifuentes-Pagano, E., et al., (2014) “The Quest for Selective Nox Inhibitors and Therapeutics: Challenges, Triumphs and Pitfalls” Antioxid Redox Signal. 20: 2741-2754, which are each incorporated by reference in its entirety. More examples of RAC1 inhibitors are disclosed in Arnst, J. L. et al., (2017) “Discovery and characterization of small molecule Rac1 inhibitors”, Oncotarget. 8: 34586-34600.

### Pharmaceutical Compositions and Formulations

Some embodiments of the methods and compositions provided herein include pharmaceutical compositions, and administration of such compositions. In some embodiments, a pharmaceutical composition can include an agent which reduces the activity of NOX2 in a subject, such as a NOX2 inhibitor. In some embodiments, a pharmaceutical composition can include an agent, such as a NOX2 inhibitor, and a pharmaceutically acceptable excipient. As used herein, a “pharmaceutically acceptable” can include a carrier, diluent or excipient that does not abrogate the biological activity and properties of a NOX2 inhibitor in a subject. Standard pharmaceutical formulation techniques can be used, such as those disclosed in Remington's The Science and Practice of Pharmacy, 21st Ed., Lippincott Williams & Wilkins (2005), incorporated by reference in its entirety.

In some embodiments, a pharmaceutical composition can be administered to a subject by any of the accepted modes of administration for agents that serve similar utilities including, but not limited to, orally, subcutaneously, intravenously, intranasally, topically, transdermally, intraperitoneally, intramuscularly, intrapulmonary, vaginally, rectally, or intraocularly.

### Kits

Some embodiments of the methods and compositions provided herein include kits comprising an agent for reducing activity of NOX2 in a subject such as a NOX2 inhibitor. Examples of NOX2 inhibitors include histamine dihydrochloride (HDC) (CEPLENE), GSK2795039, apocynin, GKT136901, GKT137831, ML171, VAS2870, VAS3947, celastrol, ebselen, perhexiline, grindelic acid, NOX2ds-tat, NOXAlds, fulvene-5, ACD 084, and shionogi. Altenhofer, S. et al., “Evolution of NADPH Oxidase Inhibitors: Selectivity and Mechanisms for Target Engagement”, Antioxid Redox Signal. 2015 23: 406-427; Hirano, K. et al., “Discovery of GSK2795039, a Novel Small Molecule NADPH Oxidase 2 Inhibitor”, Antioxid Redox Signal. 2015 23: 358-374, which are each incorporated by reference in its entirety. More examples of NOX2 inhibitors include histamine, N-methyl-histamine, 4-methyl-histamine, histamine phosphate, histamine diphosphate, idelalisib, and a class I phosphatidylinositol 3 kinase delta (PI3K delta) inhibitor. In some embodiments, the NOX2 inhibitor is HDC.

### EXAMPLES

### Example 1—In Vivo Murine Model for Surgical-Induced Inflammation

To mimic the wounding inflammation associated with surgery, sterile polyvinyl alcohol (PVA) sponges (1 cm² with a thickness of 2 mm) were implanted subcutaneously to wild type C57BL/6 and Nox2−/− mice (mouse strain B6.129S6-Cybbtm1Din, obtained from the Jackson Laboratory) aiming to induce tissue inflammation.

Mice received intraperitoneal injections of the NOX2-inhibitor HDC (1,500 μg/mouse) thrice weekly for 2 weeks starting two days before implantation of sponges.

One week after implantation of sponges, before injection of melanoma cells, peripheral blood was drawn from vena saphena of wild type and Nox2−/− mice. Blood cells were stained for CD11b and Ly6C using fluorochrome-conjugated antibodies and with DCFDA to detect content of intracellular reactive oxygen species. The content of CD11b+LyC6+inflammatory monocytes in blood and their levels of intracellular radicals was determined using flow cytometry. These levels were normalized against those of wild type control mice that did not receive sponges.

One week after implantation of sponges, B16F10 murine melanoma cells were injected intravenously (100,000 cells to wild type mice and 150,000 to Nox2−/− mice). Metastatic foci formed in lungs were enumerated macroscopically 3 weeks after injection of melanoma cells.

### Example 2—In Vivo Treatment of Subjects

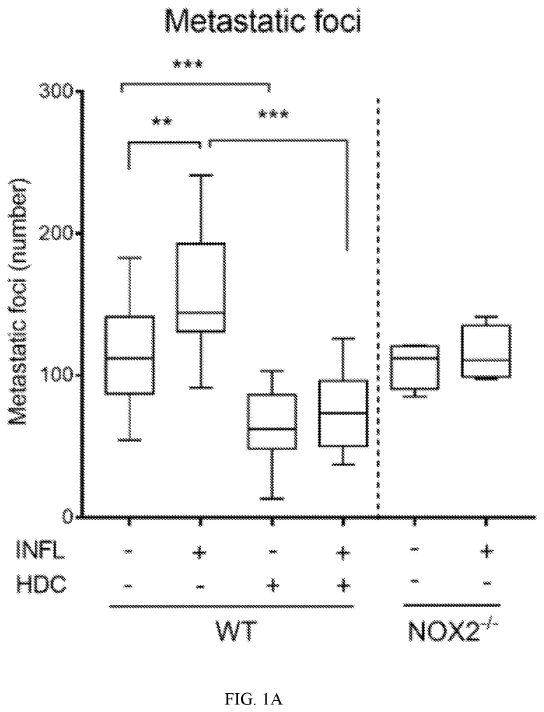

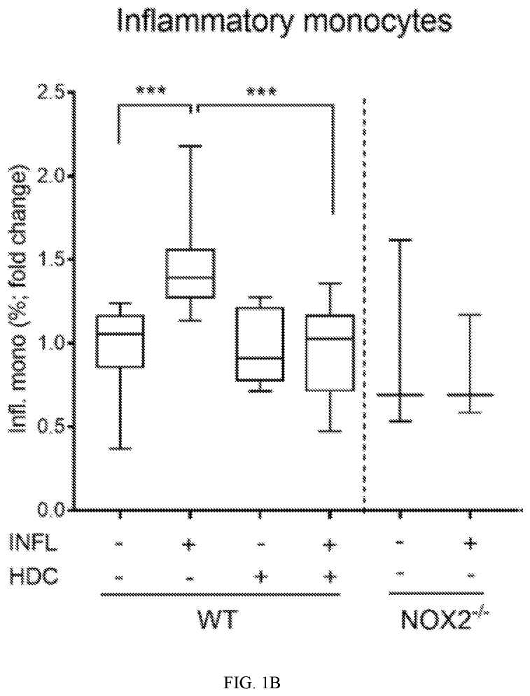

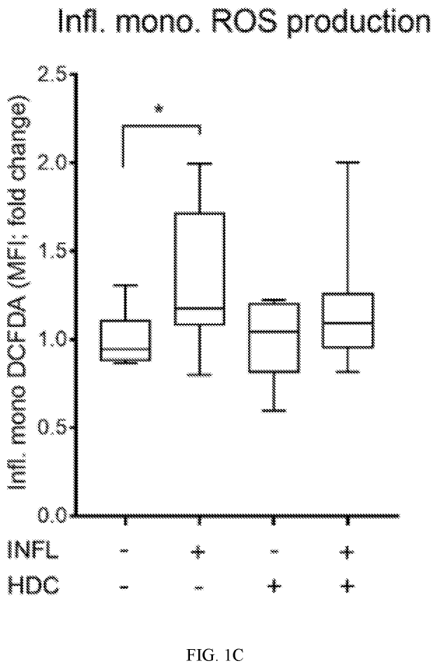

To mimic the inflammation associated with surgery, sterile PVA sponges were implanted subcutaneously to wild-type- and NOX2-deficient (Nox2−/−) mice one week before injection of B16F10 melanoma cells into the blood stream. The presence of implanted sponges increased the number of pulmonary metastases formed from the injected melanoma cells in wild-type mice (p=0.0015, n=19) but not in corresponding Nox2−/− mice (p>0.5, n=4) (FIG. 1A). Treatment with the NOX2 inhibitor histamine dihydrochloride (HDC) significantly reduced the formation of metastases during surgical inflammation in wild type mice (p<0.0001, n=10) (FIG. 1A). To determine effects of the implanted sponge on inflammation, blood was drawn from wild type and Nox2−/− mice one week after the implantation of sponges. In sponge-bearing wild type mice there was a significant increase in the frequency of CD11b+LyC6+ inflammatory monocytes (p<0.0001, n=18; FIG. 1B) and in their formation of ROS (p=0.01, n=10; FIG. 1C). No increase in inflammatory monocytes was noted in sponge-bearing Nox2−/− mice (p>0.5, n=3) (FIG. 1B). In vivo treatment with HDC completely prevented the increase in inflammatory monocytes in wounded wild type mice (p<0.0001, n=10) (FIG. 1B).

The wounding-induced inflammation triggered a significant increase in the number of metastases formed from the injected melanoma cells in wild type mice but not in corresponding Nox2−/− mice (FIG. 1A).

Treatment of mice with HDC significantly reduced the formation of melanoma metastases during wounding-induced inflammation in wild type mice (FIG. 1A).

One week after the implantation of sponges, the frequency of CD11b+LyC6+ inflammatory monocytes was significantly increased in sponge-bearing wild type mice. This increase was significantly reduced in animals treated with HDC. No increase in inflammatory monocytes in blood was observed in Nox2−/− mice following sponge implantation (FIG. 1B).

The inflammation induced by sponges in wild type mice resulted in significantly increased ROS production in blood inflammatory monocytes. There was trend towards reduced ROS levels in sponge-bearing HDC-treated wild type mice (FIG. 1C).

These data suggest that surgical wounding induces inflammation that includes augmented levels of NOX2+ inflammatory monocytes in blood. The results also imply that wounding-induced NOX2 activity, in monocytes or other NOX2-positive cells, entails extensive formation of metastases after intravenous inoculation of cancer cells (melanoma). An inhibitor of NOX2 activity, histamine dihydrochloride (HDC), reduced the degree of wounding-induced inflammation and reduced the wounding-induced enhancement of metastasis. The results point to the possibility of treating a subject who will undergo surgical removal of a cancer tumor with a NOX2 inhibitor, such as HDC, to reduce metastasis formation.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of reducing surgery-induced inflammation in a subject with cancer following surgery to remove at least one solid tumor, comprising administering an effective amount of an NADPH oxidase inhibitor to the subject.

2. (canceled)

3. The method of claim 1, further comprising the reduction of reactive oxygen species.

4. The method of claim 1, wherein the subject is human.

5. The method of claim 1 wherein the NADPH oxidase inhibitor is selected from the group consisting of histamine dihydrochloride, GSK2795039, apocynin, GKT136901, GKT137831, M1,171, VAS2870, VAS3947, celastrol, ebselen, perhexiline, grindelic acid, NOX2ds-tat, NOXAlds, fulvene-5, ACD 084, shionogi, histamine, N-methyl-histamine, 4-methy 1-histamine, histamine phosphate, histamine diphosphate a RAC1 inhibitor, a RAC2 inhibitor, NSC23766, CAS 1177865-17-6, CAS 1090893-12-1, idelaisib, and a class I phosphatidylinositol 3 kinase delta (PI3K delta) inhibitor.

6. The method of claim 1, wherein the NADPH oxidase inhibitor is histamine dihydrochloride or N-alpha-methyl-histamine dihydrochloride.

7. The method of claim 6, wherein the NADPH oxidase inhibitor is histamine dihydrochloride.

8. The method of claim 1, wherein the NADPH oxidase inhibitor is administered prior to, during or after surgery or a combination thereof.

9. The method of claim 1, wherein the cancer comprises a melanoma.

10-11. (canceled)

12. A method of reducing surgery-induced metastases in a subject with cancer following surgery to remove at least one solid tumor, comprising administering an NADPH oxidase inhibitor to the subject.

13-14. (canceled)

15. The method of claim 12, wherein the cancer comprises a melanoma.

16. A method of reducing surgery-induced inflammation in a subject, comprising: administering an effective amount of an NADPH oxidase inhibitor to the subject.

17. The method of claim 16, wherein the inflammation is reduced compared to a subject having surgery and not administered the NADPH oxidase inhibitor.

18. The method of claim 16 or 17, wherein the subject has a metastatic cancer.

19. The method of claim 18, wherein the administering an effective amount of an NADPH oxidase inhibitor to the subject is sufficient to reduce metastasis of the metastatic cancer compared to a subject having a metastatic cancer and not administered the NADPH oxidase inhibitor.

20. The method of claim 18, wherein the metastatic cancer comprises a melanoma.

21. The method of claim 16, wherein the NADPH oxidase inhibitor is administered prior to, during or after surgery or a combination thereof the surgery.

22. (canceled)

23. The method of claim 16, wherein the NADPH oxidase inhibitor is administered at least weekly.

24. The method of claim 16, wherein the NADPH oxidase inhibitor is administered at least three times weekly.

25. The method of claim 16, wherein the NADPH oxidase inhibitor is administered for at least 2 weeks.

26. The method of claim 16, wherein the subject is human.

27-30. (canceled)
